(19) United States
(12) Patent Application Publication
(10) Pub. No.: US 2008/0099357 A1
(43) Pub. Date: May 1, 2008

(54) Container for collecting and transporting special materials

(75) Inventor: Evert Cornelis Antonie Versluis, Nijkerk (NL)

Correspondence Address: WESTMAN CHAMPLIN & KELLY, P.A., SUITE 1400, 900 SECOND AVENUE SOUTH, MINNEAPOLIS, MN, 55402-3319, US

(73) Assignee: Ecama Holding B.V., Nijkerk (NL)

(21) Appl. No.: 11/586,114

(22) Filed: Oct. 25, 2006

(51) Int. Cl. A61B 19/02

(52) U.S. Cl. 206/438

(57) ABSTRACT

A container for collecting and transporting special materials, such as, for example, hospital waste and diagnostic samples, is provided with a primary holder, a secondary holder and a closed, rigid outer box. The secondary holder comprises a secondary flexible bag which can be closed in such a manner that it is at least substantially liquid-tight and airtight, and the dimensions of which, at any rate in the event of internal excess pressure, are at least equal to the internal dimensions of the rigid outer box. In the event of excess pressure in the secondary flexible bag, the flexible bag and the outer box with cover interact in order to withstand the excess pressure.

### BACKGROUND

The invention relates to a container for collecting and transporting special materials, such as, for example, hospital waste and diagnostic samples, which container comprises a primary holder, a secondary holder and a closed, rigid outer box.

Such a container is known, for example from EP-A-1 472 987. It has been found from experiments with this known container that improvement is possible as regards the closure of the container and the liquid-tightness and airtightness. The known container does not, for example, meet the standards laid down in Packing Instruction P620.

### SUMMARY

According to one aspect of the invention, the secondary holder comprises a secondary flexible bag which can be closed in such a manner that it is at least substantially liquid-tight and airtight, and the dimensions of which, at least in the event of internal excess pressure, are at least equal to the internal dimensions of the rigid outer box, wherein in the event of excess pressure in the secondary flexible bag said flexible bag and the closed outer box interact in order to withstand the excess pressure.

A container that meets very high standards as regards liquid-tightness and airtightness is obtained in this manner. Owing to the fact that the dimensions of the secondary flexible bag are such that in the event of excess pressure said bag lies against the inner of the outer box, the flexible bag can interact with the outer box in the event of excess pressure in order to withstand the excess pressure. It has been found that such a container is capable of withstanding a pressure difference of 95 kPa without any problem.

According to an advantageous embodiment, the primary holder comprises an inner box and a primary flexible bag enclosing the inner box, which inner box has a closed bottom and upper wall, the primary flexible bag being closable so that it is at least substantially liquid-tight and airtight, which primary holder is accommodated in the secondary flexible bag and is accommodated together with the secondary bag in the outer box with a close fit. A container that fully meets Packing Instruction P260 is obtained in this manner.

According to a particularly advantageous embodiment, the outer box is designed with a rectangular open upper side and a cover which, in a closure position, can be placed on the outer box in order to close the outer box, at least one locking opening being provided in each of at least two opposite side walls, near the open upper side of the outer box, which locking opening is closed off on the inside by a box part that separates the secondary bag from the locking opening, and the cover having a peripheral edge which encloses the side walls of the outer box in the closure position of the cover and which bears a locking lip at the location of each locking opening, which locking lip can be pushed into the locking opening between the box part and the side wall of the outer box. In this embodiment the locking engagement of the cover is strengthened when there is an increase in the internal pressure of the container, owing to the fact that the secondary flexible bag wedges the locking lips between the box part and the side walls of the outer box.

Another aspect of the invention is a method for packing special materials, such as, for example, hospital waste and diagnostic samples, wherein the special material is placed in the liquid-tight primary holder of a container according to the invention, which method includes closing the flexible secondary bag so as to be at least substantially liquid-tight and airtight, and the rigid outer box is then closed by pressing the cover onto the outer box in the closure position, after which each locking lip of the cover is pushed into the corresponding locking opening between the box part and the side wall of the outer box.

### BRIEF DESCRIPTION OF THE DRAWINGS

Aspects of the invention will be explained in greater detail below with reference to the drawing, in which some embodiments of the container are schematically shown.

FIG. 1 is a perspective view of a first embodiment of the container, in which the various parts of the container are shown in the disassembled state.

FIG. 2 is a schematic cross-sectional view of the container of FIG. 1, in the assembled state.

FIG. 3 shows the primary holder of the container of FIG. 1, with the primary flexible bag closed so as to be liquid-tight and airtight.

FIG. 4 shows the intermediate box of the container of FIG. 1, in the open state.

FIG. 5 is a perspective view of the outer box with the corresponding cover of the container of FIG. 1.

FIGS. 6A and 6B show schematically a locking lip and the placing of said locking lip in the locking opening.

FIG. 7 shows the situation in which the locking lip is fully inserted into the locking opening.

FIG. 8 shows an alternative embodiment of the container.

### DETAILED DESCRIPTION

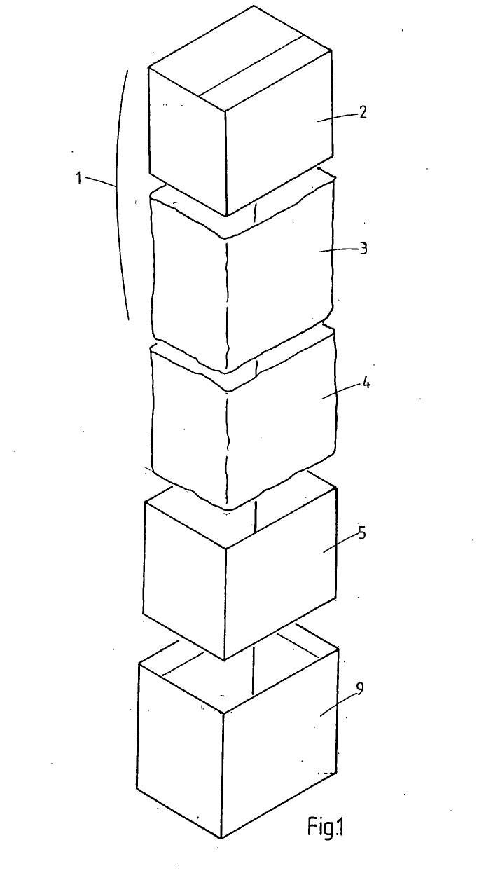

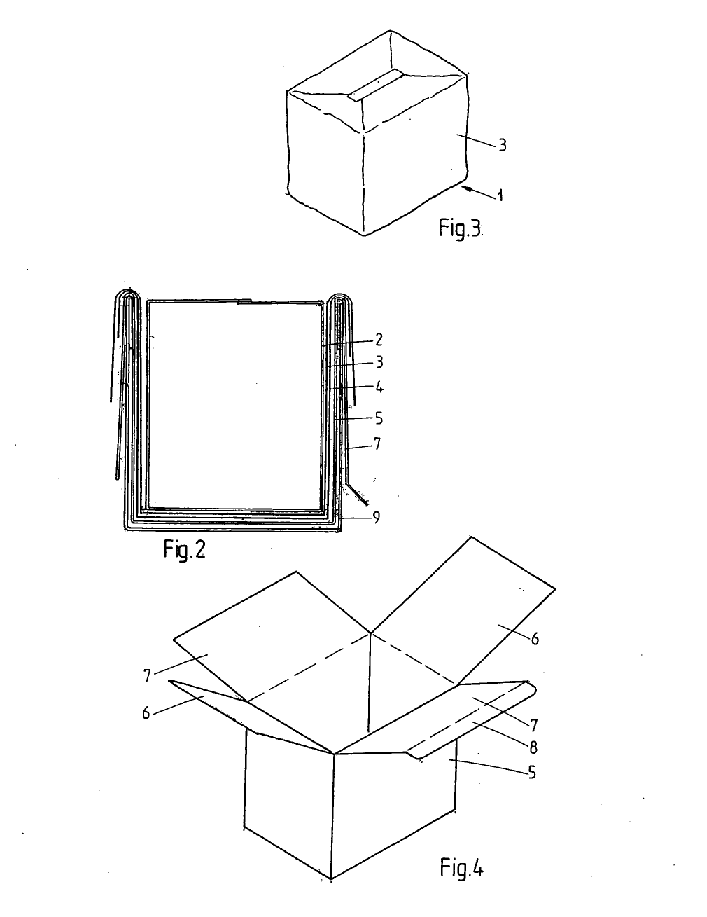

FIG. 1 shows an embodiment of a container for collecting and transporting special materials, such as, for example, hospital waste and diagnostic samples, in the disassembled state. FIG. 2 shows said container in cross section. The container shown is provided with a primary holder 1, which in the case of this embodiment includes an inner box 2 and a primary flexible bag 3. Said primary flexible bag is made of a suitable material, such as, for example, latex, neoprene, chloroprene, PVC, HDPE, LDPE or the like, which is at least virtually airtight and liquid-tight, and which can also be closed in a suitable manner so as to be airtight and liquid-tight. This closure can be achieved, for example, by thermal welding or gluing, or by using suitable mechanical closure devices such as tape. FIG. 3 shows as an example the closed primary holder 1, with a schematically indicated mechanical closure means which provides an airtight and liquid-tight closure of the primary flexible bag 3.

The inner box 2 has an upper side and a bottom side with four flaps, the long flaps overlapping each other by several centimetres. A box with a closed top and bottom is obtained by folding the flaps in the plane of the upper and bottom sides. The material of the inner box 2 is of waterproof cardboard, for example SAPINO waterproof cardboard, 600 g/m². Of course, a different grade of cardboard or a different material can also be used, depending on the envisaged use of the container and the standards that have to be met by the container. The inner box 2 ensures that the primary holder 1 is puncture resistant, while it is ensured by means of the primary flexible bag 3 that the primary holder can be closed so as to be liquid-tight and airtight.

The primary holder 1, comprising the inner box 2 and the primary bag 3, is placed in a secondary holder 4, which in the embodiments described is in the form of a secondary flexible bag, which in the embodiments described meets the same specifications as the primary flexible bag. A suitable absorbent material (not shown) can be placed between the bags 3 and 4, which absorbent material absorbs escaping moisture in the event of leakage of the primary holder.

In the embodiment according to FIG. 1 the assembly comprising primary holder 1 and secondary holder 4 is placed in an intermediate box 5, which is shown in greater detail in FIG. 4. The intermediate box 5 has a rectangular cross section and has top flaps 6, 7 and corresponding bottom flaps, which are not visible in FIG. 4. Each flap 6, 7 has such dimensions that it fully covers the opening of the intermediate box 5, one of the flaps 7 also being provided with a closure flap 8, which during closure of the container falls between the opposite wall of an outer box 9 shown in FIG. 1 and the outer side of the intermediate box 5. In the case of the embodiment described the intermediate box 5 is made of a waterproof cardboard, for example SAPINO waterproof cardboard, 1100 g/m². Of course, a different grade of cardboard or a different material can also be used, depending on the envisaged use of the container and the standards that have to be met by the container.

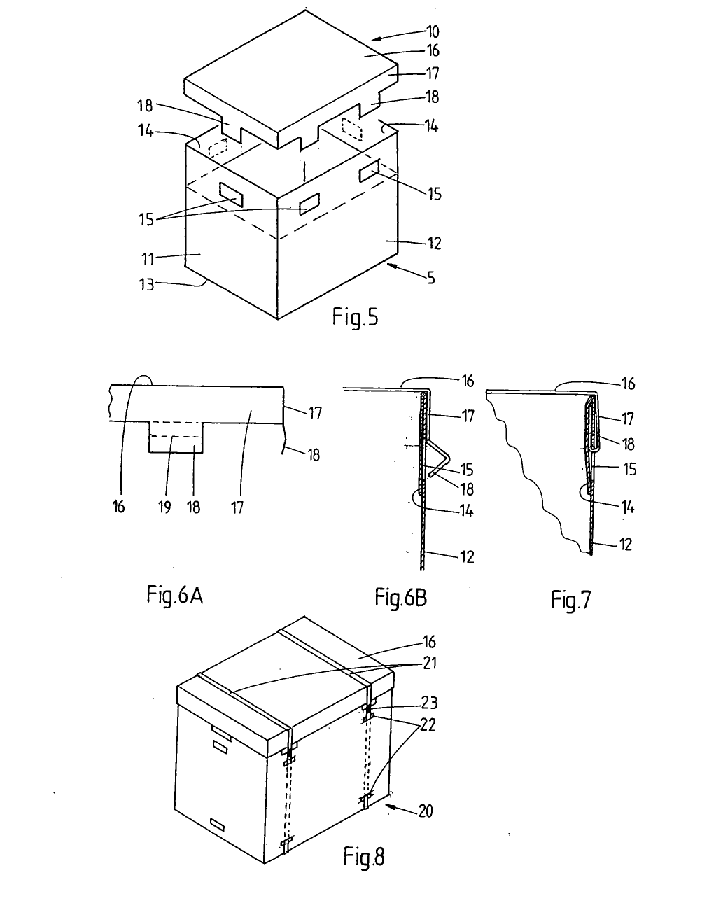

The intermediate box 5 with the primary holder 1 and secondary holder 4 placed therein are placed in the outer box 9, the intermediate box 5 being accommodated in the outer box 9 with a close fit. A schematic cross section of the assembled container is shown in FIG. 2. The outer box 9 and a corresponding cover 10 are illustrated in greater detail in FIG. 5, while FIGS. 6 and 7 show a number of details. The outer box 9 and the cover 10 in this case are made of the same material as the intermediate box 5 and, depending on the envisaged use, may be made of a heavier or a lighter grade of cardboard.

The outer box 9 has pairs of opposite side walls 11, 12, a closed bottom 13 and an open upper side. At the upper side the side walls have top flaps 14, which are folded inwards and lie against the inner side of the side walls 11, 12. A locking opening 15 is provided in each of the two short side walls 11, while two locking openings 15 are present in the two opposite long side walls 12. The locking openings 15 are covered by the uppers flaps 14 when the latter are folded inwards.

The cover 10 has a peripheral edge 17, which projects downwards from the upper surface 16 and which, in the closure position of the cover that is partially shown in FIG. 7, encloses the side walls 11, 12. At the location of each locking opening 15, the peripheral edge 17 bears a locking lip 18, which can be pushed into the corresponding locking opening 15 between the downwardly folded top flap 14 and the side wall 11, 12 respectively of the outer box 9.

In the embodiment as shown, a fold line 19 is formed in each locking lip 18, in order to make it easier to push a locking lip 18 into the locking opening 15, as shown schematically in FIGS. 6A and 6B. If desired, two or more fold lines 19 may be disposed at regular intervals over the height of the locking lip 18.

In the container described with reference to FIG. 1-7, the dimensions and/or the properties of the material of at least the secondary flexible bag are selected in such a manner that in the event of any excess pressure inside said bag 4, said bag 4 will lie with its entire external surface against the inner side of the intermediate box 5. Said intermediate box 5 in turn is placed in the outer box 9 with a close fit, so that in the event of increasing pressure the boxes 5, 9 will provide the mechanical strength to resist the excess pressure. The secondary flexible bag therefore provides the required airtight and liquid-tight properties, while the boxes 5, 9 deliver the required strength. In other words, in the event of excess pressure in the secondary flexible bag 4, said bag will interact with the intermediate box 5 and the outer box 9 to withstand the excess pressure. The primary bag 3 preferably has the same properties as the secondary bag 4, so that in the event of excess pressure in the primary bag, both bags will interact with the boxes 5 and 9, each of the two bags 3, 4 being capable of withstanding the excess pressure by interacting with the boxes 5, 9.

The above-described locking of the cover 10 on the outer box 9 with the aid of the locking lips 18 and locking openings 15 furthermore has the advantage that in the event of an increase in the internal pressure in the container the top flaps 14 will clamp the inserted locking lips 18 against the side walls 11, 12, with the result that the clamping force increases as the pressure increases.

In experiments carried out with the container as described herein it was found that an excess pressure of 95 kPa gives rise to no damage whatsoever. Moreover, the container described withstands the usual drop tests and bar tests that containers of this type must pass.

In an alternative embodiment the container may be produced without the intermediate box if the outer box 9 has sufficient strength for the purpose for which the container is intended. In the embodiment with the intermediate box 5 as described above, the top flaps 14 of the outer box 9 may be dispensed with if desired, since the vertical walls of the intermediate box 5 close off the locking openings 15. The locking lips 18 are pushed between the walls of the intermediate box 5 and the outer box 9 in this case, and with increasing pressure in the container are wedged between the walls of the intermediate box 5 and the outer box 9. In both variants it is therefore a box part, i.e. the vertical wall of the intermediate box 5 or the folded-over top flap 14 of the outer box 9, that separates the locking opening 15 from the secondary bag 4 and that clamps the locking lip 18 against the side wall 11, 12. This means that in both variants the outer box 9 is puncture resistant.

According to an advantageous embodiment of the invention, at least the secondary flexible bag 4, and preferably also the primary flexible bag 3, is so dimensioned that in the event of excess pressure of, for example, 95 kPa, if the intermediate box 5 and outer box 9 will give way, said bag or bags can expand, without becoming damaged, in order to lower the pressure inside the secondary flexible bag 4. As an alternative, the elasticity of the secondary and/or primary bag 4, 3 can also be such that said bag or bags can expand a number of times in order to lower the internal pressure, without giving way. It is further possible to achieve an expansion of the flexible bag by a combination of the dimensions and elasticity. By using a flexible bag 3, 4 which meets these standards it is ensured that if the outer box 9 and the intermediate box 5 unexpectedly give way, the bag 3, 4 can expand without giving way.

FIG. 8 shows in perspective view an alternative embodiment of the container, which is designed in substantially the same way as the container according to FIG. 1. In this case an outer box 20 provided with closure tapes 21, shown schematically in FIG. 8, is used. The closure tapes in this case extend over the locking lips 18 and locking openings 15 in the long side walls 12 of the outer box 9. If desired, the cover 10 can be designed without locking lips 18 and the outer box 9 can be designed without locking openings 15 respectively in this embodiment. As indicated by a dashed line in FIG. 8, the closure tapes 21 are guided partially along the inner side of the long side walls 12, for which purpose grooves 22 have been provided in said side walls 12. Each closure tape 21 has a closure element such as a clasp or the like, which is indicated schematically by 23 in FIG. 8.

In the embodiments described the cover 10 is fixed on the outer box 9, 20 by the locking lips 18 alone or by the closure tapes as well. As an alternative, it is also possible to provide an adhesive strip (not shown) on the inner side of the cover 10 along the peripheral edge 17, against which adhesive strip the top edge of the outer box 9, 20 can be pressed.

It will be apparent from the foregoing that the invention provides a pressure-resistant container, which can withstand high internal pressures of 95 kPa or more. Furthermore, the container described has a liquid-tight and airtight primary holder 1 and a liquid-tight and airtight secondary holder 4. The primary holder 4 is also puncture resistant. The selected dimensions and/or the material properties enable at least said secondary holder 4, and preferably also the primary bag 3, to interact with the outer box 9, possibly with the interposition of an intermediate box 5, in such a manner that the container can withstand a high internal pressure.

The invention is not restricted to the embodiments described above, which can be varied in several ways, for example in the shape of the boxes and corresponding covers, without departing from the scope of the claims. In addition, aspects of the container(s) as described in European Patent Application 06110141.6, published Aug. 23, 2006 as EP1693019A2, which is hereby incorporated by reference in its entirety, can be adopted, if desired.

1. A container for collecting and transporting special materials, such as, for example, hospital waste and diagnostic samples, which container comprises a primary holder, a secondary holder and a closed, rigid outer box, the secondary holder comprises a secondary flexible bag which can be closed in such a manner that it is at least substantially liquid-tight and airtight, and the dimensions of which, at least in the event of internal excess pressure, are at least equal to the internal dimensions of the rigid outer box, wherein in the event of excess pressure in the secondary flexible bag said flexible bag and the closed outer box interact in order to withstand the excess pressure, wherein the primary holder comprises an inner box and a primary flexible bag enclosing the inner box, which inner box has a closed bottom and upper wall, the primary flexible bag being closable so that it is at least substantially liquid-tight and airtight, which primary holder is accommodated in the secondary flexible bag and is accommodated together with the secondary bag in the outer box with a close fit.

2. A container according to claim 1, wherein the primary holder accommodated in the secondary flexible bag is placed in an intermediate box, which is accommodated in the outer box with a close fit.

3. A container according to claim 2, wherein the intermediate box is provided with top and bottom flaps, which each fully cover the rectangular opening surface of the intermediate box, wherein a top flap preferably has a closure lip which can be inserted between the outer side of the intermediate box and the inner side of the outer box.

4. A container according to claim 1, wherein the outer box is designed with a rectangular open upper side and a cover which, in a closure position, can be placed on the outer box in order to close the outer box, at least one locking opening being provided in each of at least two opposite side walls, near the open upper side of the outer box, which locking opening is closed off on the inside by a box part that separates the secondary bag from the locking opening, and the cover having a peripheral edge which, in the closure position of the cover, encloses the side walls of the outer box and which, at the location of each locking opening, bears a locking lip, which locking lip can be pushed into the locking opening between the box part and the side wall of the outer box.

5. A container according to claim 4, wherein an adhesive strip is provided on the inner side of the cover near the peripheral edge, wherein the top edge of the outer box projects into the adhesive strip if the cover is pressed into the closure position on the outer box.

6. A container according to claim 4, wherein each locking lip is provided with one or more fold lines.

7. A container according to claim 1, wherein the dimensions and/or the elasticity of the primary and/or secondary flexible bag are selected in such a manner that in the absence of the outer box an expansion of the primary and/or secondary bag is possible in order to release excess pressure in the primary and/or secondary bag.

8. A container according to claim 7, wherein the dimensions and/or elasticity of the primary and/or secondary flexible bag permit an expansion of at least five times their size.

9. A container according to claim 1, wherein one or more closure tapes are provided, which closure tapes enclose the outer box and the cover placed in the closure position, each closure tape preferably running over locking openings provided in opposite side walls.

10. A container according to claim 9, wherein the closure tapes run over part of the height of the side walls of the outer box on the inner side thereof.

11. A container according to claim 1, wherein the inner box of the primary holder is provided with flattened box corner points.

12. A container according to claim 1, wherein a liquid-absorbing material is inserted between the primary and secondary holders.

13. A method for packing special materials, such as, for example, hospital waste and diagnostic samples, wherein the special material is placed in the liquid-tight primary holder of a container according to one of the preceding claims, wherein the flexible secondary bag is closed so as to be at least substantially liquid-tight and airtight and the rigid outer box is then closed by pressing the cover onto the outer box in the closure position, after which each locking lip of the cover is pushed into the corresponding locking opening between the box part and the side wall of the outer box.

14. A method according to claim 13, wherein the primary holder comprises an inner box and a flexible bag enclosing the inner box, wherein prior to the closure of the secondary flexible bag the primary flexible bag is closed in such a manner that it is at least substantially liquid-tight and airtight.

15. A method according to claim 14, wherein the primary holder accommodated in the secondary flexible bag is placed in an intermediate box, and wherein after closure of the secondary bag the top flaps of the intermediate box are closed and the closure lip is inserted between the outer side of the intermediate box and the inner side of the outer box.
